(19) United States
(12) Patent Application Publication
(10) Pub. No.: US 2006/0094911 A1
(43) Pub. Date: May 4, 2006

(54) Noncatalytic manufacture of 1,1,3,3,3-pentafluoropropene from 1,1,1,3,3,3-hexafluoropropane

(76) Inventors: Velliyur Nott Mallikarjuna Rao, Wilmington, DE (US); Allen Capron Sievert, Elkton, MD (US); Ralph Newton Miller, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/259,901

(22) Filed: Oct. 27, 2005

Related U.S. Application Data
(60) Provisional application No. 60/623,210, filed on Oct. 29, 2004.

(51) Int. Cl. C07C 17/25

(52) U.S. Cl. 570/155

(57) ABSTRACT

1,1,3,3,3-Pentafluoropropene (CF₃CH═CF₂, HFC-1225zc) can be produced by pyrolyzing 1,1,1,3,3,3-hexafluoropropane (CF₃CH₂CF₃, HFC-236fa) in the absence of dehydrofluorination catalyst at temperatures of from about 700° C. to about 1000° C. and total pressures of about atmosphere pressure in an empty, tubular reactor, the interior surfaces of which comprise materials of construction resistant to hydrogen fluoride.

### BACKGROUND OF THE INVENTION

1. Field of the Invention.

This invention relates to a process for the production of 1,1,3,3,3-pentafluoropropene (CF₃CH═CF₂ or HFC-1225zc) by the thermal elimination of hydrogen fluoride from 1,1,1,3,3,3-hexafluoropropane (CF₃CH₂CF₃ or HFC-236fa). This invention further relates to azeotropic and azeotrope-like compositions comprising hydrogen fluoride and 1,1,3,3,3-pentafluoropropene, as well as azeotropic distillation processes for separating said compositions.

2. Description of Related Art.

1,1,3,3,3-Pentafluoropropene is a useful cure-site monomer in polymerizations to form fluoroelastomers. U.S. Pat. No. 6,703,533, 6,548,720, 6,476,281, 6,369,284, 6,093,859, and 6,031,141, as well as published Japanese patent applications JP 09095459 and JP 09067281, and WIPO publication WO 2004018093, disclose processes wherein 1,1,1,3,3,3-hexafluoropropane is heated at temperatures below 500° C. in the presence of catalyst to form 1,1,3,3,3-pentafluoropropene. These low-temperature catalytic routes are chosen because of the well-known tendency for fluorocarbons to fragment at higher temperatures, e.g., above 500° C. This is made clear in Chemistry of Organic Fluorine Compounds, by Milos Hudlicky, 2^{nd} Revised Edition, Ellis Horwood PTR Prentice Hall [1992] p. 515: “Polyfluoroparaffins and especially fluorocarbons and other perfluoro derivates show remarkable heat stability. They usually do not decompose at temperatures below 300° C. Intentional decomposition, however, carried out at temperatures of 500-800° C., causes all possible splits in their molecules and produces complex mixtures which are difficult to separate.”

US patent application publication US2002/0032356 discloses a process for producing the perfluorinated monomers tetrafluoroethylene and hexafluoropropylene in a gold-lined pyrolysis reactor.

The catalytic process has disadvantages, including catalyst preparation, start-up using fresh catalyst, catalyst deactivation, potential for plugging of catalyst-packed reactors with polymeric by-products, catalyst disposal or reactivation, and long reaction times that impose a space/time/yield reactor penalty. It would be desirable to be able to produce 1,1,3,3,3-pentafluoropropene from 1,1,1,3,3,3-hexafluoropropane in high yield by a non-catalytic process.

### BRIEF SUMMARY OF THE INVENTION

The present invention provides a process for producing CF₃CH═CF₂ in the absence of dehydrofluorination catalyst. In particular, the process of the present invention comprises pyrolyzing CF₃CH₂CF₃ to make CF₃CH═CF₂. Pyrolyzing accomplishes the thermal decomposition of the CF₃CH₂CF₃, at a temperature greater than about 700° C.

This selective formation of CF₃CH═CF₂ embodies several unexpected results. First, it is surprising that the heat input of the pyrolysis process does not cause the CF₃CH₂CF₃ reactant to fragment to C-1, e.g., methanes, and C-2, e.g., ethane and ethylene, compounds. Second, it is surprising that the CF₃CH═CF₂ product is stable under pyrolysis conditions and does not undergo further conversion to rearranged products or to products containing fewer hydrogen and/or fluorine atoms. Third, it is surprising that the pyrolysis to form CF₃CH═CF₂ takes place with high selectivity.

### DETAILED DESCRIPTION OF THE INVENTION

The present invention provides a process of producing CF₃CH═CF₂ by pyrolysis of CF₃CH₂CF₃. The process may be written as:
CF₃CH₂CF₃+Δ→CF₃CH═CF₂+HF
where Δ represents heat and HF is hydrogen fluoride.

Pyrolysis, as the term is used herein, means chemical change produced by heating in the absence of catalyst. Pyrolysis reactors generally comprise three zones: a) a preheat zone, in which reactants are brought close to the reaction temperature; b) a reaction zone, in which reactants reach reaction temperature and are at least partially pyrolyzed, and products and any byproducts form; c) a quench zone, in which the stream exiting the reaction zone is cooled to stop the pyrolysis reaction. Laboratory-scale reactors have a reaction zone, but the preheating and quenching zones may be omitted.

The reactor may be of any shape consistent with the process but is preferably a cylindrical tube, either straight or coiled. Although not critical, such reactors typically have an inner diameter of from about 1.3 to about 5.1 cm (about 0.5 to about 2 inches). Heat is applied to the outside of the tube, the chemical reaction taking place on the inside of the tube. The reactor and its associated feed lines, effluent lines and associated units should be constructed, at least as regards the surfaces exposed to the reaction reactants and products, of materials resistant to hydrogen fluoride. Typical materials of construction, well-known to the fluorination art, include stainless steels, in particular of the austenitic type, the well-known high nickel alloys, such as Monel® nickel-copper alloys, Hastelloy-based alloys and Inconel® nickel-chromium alloys and copper clad steel. Where the reactor is exposed to high temperature the reactor may be constructed of more than one material. For example, the outer surface layer of the reactor should be chosen for ability to maintain structural integrity and resist corrosion at the pyrolysis temperature, the inner surface layer of the reactor should be chosen of materials resistant to attack by, that is, inert to, the reactant and products. In the case of the present process, the product hydrogen fluoride is corrosive to certain materials. In other words, the reactor may be constructed of an outer material chosen for physical strength at high temperature and an inner material chosen for resistance to corrosion by the reactants and products under the temperature of the pyrolysis.

For the process of this invention, it is preferred that the reactor inner surface layer be made of high nickel alloy, that is an alloy containing at least about 50 wt % nickel, preferably a nickel alloy having at least about 75 wt % nickel, more preferably a nickel alloy having less than about 8 wt % chromium, still more preferably a nickel alloy having at least about 98 wt % nickel, and most preferably substantially pure nickel, such as the commercial grade known as Nickel 200. More preferable than nickel or its alloys as the material for the inner surface layer of the reactor is gold. The thickness of the inner surface layer does not substantially affect the pyrolysis and is not critical so long as the integrity of the inner surface layer is intact. The thickness of the inner surface layer is typically from about 10 to about 100 mils (0.25 to 2.5 mm). The thickness of the inner surface layer can be determined by the method of fabrication, the cost of materials, and the desired reactor life.

The reactor outer surface layer is resistant to oxidation or other corrosion and maintains sufficient strength at the reaction temperatures to keep the reaction vessel from failing of distorting. This layer is preferably Inconel® alloy, more preferably Inconel® 600.

The present pyrolysis of CF₃CH₂CF₃ to CF₂═CHCF₃ and HF is carried out in the absence of catalyst in a substantially empty reactor. By absence of catalyst is meant that no material or treatment is added to the pyrolysis reactor that increases the reaction rate by reducing the activation energy of the pyrolysis process. It is understood that although surfaces that are unavoidably present in any containment vessel, such as a pyrolysis reactor, may have incidental catalytic or anticatalytic effects on the pyrolysis process, the effect makes an insignificant contribution, if any, to the pyrolysis rate. More specifically, absence of catalyst means absence of conventional catalysts having high surface area in a particulate, pellet, fibrous or supported form that are useful in promoting the elimination of hydrogen fluoride from a hydrofluorocarbon (i.e., dehydrofluorination). For example, dehydrofluorination catalysts include: chromium oxide, optionally containing other metals, metal oxides or metal halides; chromium fluoride, unsupported or supported; and activated carbon, optionally containing other metals, metal oxides or metal halides.

Substantially empty reactors useful for carrying out the present process are tubes comprising the aforementioned materials of construction. Substantially empty reactors include those wherein the flow of gases through the reactor is partially obstructed to cause back-mixing, i.e. turbulence, and thereby promote mixing of gases and good heat transfer. This partial obstruction can be conveniently obtained by placing packing within the interior of the reactor, filling its cross-section or by using perforated baffles. The reactor packing can be particulate or fibrillar, preferably in cartridge disposition for ease of insertion and removal, has an open structure like that of Raschig Rings or other packings with a high free volume, to avoid the accumulation of coke and to minimize pressure drop, and permits the free flow of gas. Preferably the exterior surface of such reactor packing comprises materials identical to those of the reactor inner surface layer; materials that do not catalyze dehydrofluorination of hydrofluorocarbons and are resistant to hydrogen fluoride. The free volume is the volume of the reaction zone minus the volume of the material that makes up the reactor packing. The free volume is at least about 80%, preferably at least about 90%, and more preferably about 95%.

The pyrolysis which accomplishes the conversion of CF₃CH₂CF₃ to CF₂═CHCF₃ is suitably conducted at a temperature of at least about 700° C., preferably at least about 750° C., and more preferably at least about 800° C. The maximum temperature is no greater than about 1000° C., preferably no greater than about 950° C., and more preferably no greater than about 900° C. The pyrolysis temperature is the temperature of the gases inside at about the mid-point of the reaction zone.

The residence time of gases in the reaction zone is typically from about 0.5 to about 60 seconds, more preferably from about 2 seconds to about 20 seconds at temperatures of from about 700 to about 900° C. and atmospheric pressure. Residence time is determined from the net volume of the reaction zone and the volumetric feed rate of the gaseous feed to the reactor at a given reaction temperature and pressure, and refers to the average amount of time a volume of gas remains in the reaction zone.

The pyrolysis is preferably carried out to a conversion of the CF₃CH₂CF₃ at least about 25%, more preferably to at least about 35%, and most preferably to at least about 45%. By conversion is meant the portion of the reactant that is consumed during a single pass through the reactor. Pyrolysis is preferably carried out to a yield of CF₃CH═CF₂ of at least about 50%, more preferably at least about 60%, and most preferably at least about 75%. By yield is meant the moles of CF₃CH═CF₂ produced per mole of CF₃CH₂CF₃ consumed.

The reaction is preferably conducted at subatmospheric, or atmospheric total pressure. That is, the reactants plus other ingredients are at subatmospheric pressure or atmospheric pressure. (If inert gases are present as other ingredients, as discussed below, the sum of the partial pressures of the reactants plus such ingredients is subatmospheric or atmospheric). Near atmospheric total pressure is more preferred. The reaction can be beneficially run under reduced total pressure (i.e., total pressure less than one atmosphere).

The reaction according to this invention can be conducted in the presence of one or more unreactive diluent gases, that is diluent gases that do not react under the pyrolysis conditions. Such unreactive diluent gases include the inert gases nitrogen, argon, and helium. Fluorocarbons that are stable under the pyrolysis conditions, for example, trifluoromethane and perfluorocarbons, may also be used as unreactive diluent gases. It has been found that inert gases can be used to increase the conversion of CF₃CH₂CF₃ to CF₃CH═CF₂. Of note are processes where the mole ratio of inert gas to CF₃CH₂CF₃ fed to the pyrolysis reactor is from about 5:1 to 1:1. Nitrogen is a preferred inert gas because of its comparatively low cost.

The present process produces a 1:1 molar mixture of HF and CF₃CH═CF₂ in the reactor exit stream. The reactor exit stream can also contain unconverted reactant, CF₃CH₂CF₃. The components of the reactor exit stream can be separated by conventional means, such as distillation. Hydrogen fluoride and CF₃CH═CF₂ form a homogenous low-boiling azeotrope containing about 60 mole percent CF₃CH═CF₂. The present process reactor exit stream can be distilled and the low-boiling HF and CF₃CH═CF₂ azeotrope taken off as a distillation column overhead stream, leaving substantially pure CF₃CH₂CF₃ as a distillation column bottom stream. Recovered CF₃CH₂CF₃ reactant may be recycled to the reactor. CF₃CH═CF₂ can be separated from its azeotrope with HF by conventional procedures, such as pressure swing distillation or by neutralization of the HF with caustic.

The present invention further comprises azeotropic and azeotrope-like compositions comprising HF and CF₃CH═CF₂. These azeotropes contain about 60 mole percent of CF₃CH═CF₂.

The present invention further comprises a process for separating HF from a first mixture comprising HF and CF₃CH═CF₂ wherein the amount of HF in the first mixture is in excess of the amount of HF in an azeotropic or azeotrope-like composition comprising HF and CF₃CH═CF₂, comprising: distilling the first mixture to form a second mixture comprising an azeotropic or azeotrope-like composition comprising HF and CF₃CH═CF₂; recovering the second mixture as a distillation column overhead stream, and; recovering HF as a distillation column bottom stream.

The present invention further comprises a process for separating CF₃CH═CF₂ from a first mixture comprising HF and CF₃CH═CF₂ wherein the amount of CF₃CH═CF₂ in the first mixture is in excess of the amount of CF₃CH═CF₂ in an azeotropic or azeotrope-like composition comprising HF and CF₃CH═CF₂, comprising: distilling the first mixture to form a second mixture comprising an azeotropic or azeotrope-like composition comprising HF and CF₃CH═CF₂; recovering the second mixture as a distillation column overhead stream, and; recovering CF₃CH═CF₂ as a distillation column bottom stream.

Without further elaboration, it is believed the one skilled in the art can, using the description herein, utilize the present invention to the fullest extent. The following specific embodiments are to be construed as illustrative, and not as constraining the remainder of the disclosure in any way whatsoever.

### EXAMPLES

The following Examples use one of three reactors:

Reactor A: Inconel® 600 tube (this alloy is about 76 wt % nickel), 18 in (45.7 cm) long×1.0 in (2.5 cm) outer diameter×0.84 in (2.1 cm) inner diameter. Tube wall thickness is 0.16 in (0.41 cm). The preheat zone is 7 in (17.8 cm) long. The reaction zone is 2 in (5.1 cm) long. The quench zone is 7 in (17.8 cm) long. The tube is heated with 1 in (2.5 cm) diameter ceramic band heaters. The leads of a 7-point thermocouple are distributed long the length of the tube, with some in the middle of the reactor zone (to measure gas temperature).

Reactor B: Schedule 80 Nickel 200 tube with an Inconel® 617 overlay, 18 in (45.7 cm) long, 1.5 in (3.8 cm) outer diameter, 0.84 in (2.1 cm) inner diameter. The reaction zone is 2 in (5.1 cm) long. The reactor zone is heated with an 8.5 in (21.6 cm) long×2.5 in (6.35 cm) split tube furnace. The leads of a 7-point thermocouple are distributed long the length of the tube, with some in the middle of the reactor zone (to measure gas temperature).

Reactor C: Hastelloy® C276 with gold lining. Length 5 in (12.7 cm)×0.50 in (1.3 cm) outer diameter×0.35 in (0.89 cm) inner diameter. The wall thickness is 0.15 in (3.8 mm). The thickness of the gold lining is 0.03 in (0.08 cm). The reactor zone is 2 in (5.1 cm) long and is heated with a ceramic band heater.

### Example 1

Reactor A (Inconel® 600 reaction surface) is used. The reactor inlet gas temperature (“Reactor Inlet T Gas” in Table 1) is the reaction temperature. Two runs are made at reaction temperatures of 724° C. and 725° C., respectively. In Run A, the reactant feed is undiluted with inert gas. In Run B, helium and reactant are fed in the ratio of 1.4:1. The benefit of the inert gas diluent is seen in the improved yield of Run B (80%) over that of Run A (71%). A lower concentration of fluorocarbon byproducts are made in Run B. Results are summarized in Table 1. Note that “sccm” in the table stands for “standard cubic centimeters per minute”.

TABLE 1

| | |
|---|---|
| A | B |

Reactor Conditions, Feeds

| | | | | |
|---|---|---|---|---|
| Preheat Control T setting | 700° | C. | 700° | C. |
| Preheat Gas T 1″ | 545° | C. | 572° | C. |
| Preheat Gas T 2″ | 635° | C. | 655° | C. |
| Preheat Gas T 3″ | 690° | C. | 696° | C. |
| Preheat Gas T 4″ | 718° | C. | 720° | C. |
| Reactor Control T setting | 700° | C. | 700° | C. |
| Reactor Inlet T wall | 711° | C. | 710° | C. |
| Reactor Middle T wall | 700° | C. | 700° | C. |
| Reactor Exit T wall | 622° | C. | 623° | C. |
| Reactor Inlet T gas | 724° | C. | 725° | C. |
| Reactor Middle T gas | 714° | C. | 716° | C. |
| Reactor Exit T gas | 675° | C. | 673° | C. |
| HFC-236fa Feed sccm | 25 | sccm | 25 | sccm |
| Helium Feed sccm | 0 | sccm | 35 | sccm |
| Residence Time in Reaction Zone | 42 | | 18 | |
| (seconds) | | | | |

Gas Chromatograph Results in Mole %

| | | |
|---|---|---|
| CHCF₃ (HFC-23) | 4.5 | 2.1 |
| CF₃CH═CF₂ (HFC-1225zc) | 51.6 | 47.7 |
| Octafluorocyclobutane (PFC-C318) | 1.8 | 2.0 |
| CF₃CH₂CF₃ (HFC-236fa) | 27.5 | 40.3 |
| C₄H₂F₆ (HFC-1336) | 1.8 | 0.7 |
| C₄HF₇ (HFC-1327) | 1.7 | 1.2 |
| C₄HF₉ (HFC-329) | 4.2 | 2.1 |
| Other | 3.1 | 2.1 |
| Unknown | 3.8 | 1.8 |
| Conversion (%) | 72.5 | 59.7 |
| Yield (%) | 71 | 80 |

### Example 2

Reactor A (Inconel® 600 reaction surface) is used in this study of the effect of temperature on conversion and yield. Run A is made at reactor temperature of 600° C. Runs B and C are made at 699° C. and 692° C., respectively. Runs A and B are diluted 4:1 with helium. Run C is undiluted. Run A (600° C) conversion is low at 0.3%. Runs B and C (690-700° C.) have higher conversion, though still low compared to the conversion seen in Example 1, which was run at 725° C. and appreciably longer reaction zone residence times. Yields are reported, however are not reliable for such low conversions. The dependence of conversion on temperature and reaction zone residence time is plain from these experiments. Results are summarized in Table 2.

TABLE 2

| | | |
|---|---|---|
| A | B | C |

Reactor Conditions, Feeds

| | | | |
|---|---|---|---|
| Preheat Control T setting | 600 | 700 | 700 |
| (° C.) | | | |
| Preheat Gas T 1″ (° C.) | 417 | 497 | 443 |
| Preheat Gas T 2″ (° C.) | 511 | 604 | 546 |
| Preheat Gas T 3″ (° C.) | 563 | 660 | 623 |
| Preheat Gas T 4″ (° C.) | 592 | 691 | 676 |
| Reactor Control T setting | 601 | 700 | 700 |
| (° C.) | | | |
| Reactor Inlet T wall (° C.) | 615 | 718 | 722 |
| Reactor Middle T wall (° C.) | 601 | 700 | 700 |
| Reactor Exit T wall (° C.) | 566 | 661 | 653 |
| Reactor Inlet T gas (° C.) | 600 | 699 | 692 |
| Reactor Middle T gas (° C.) | 588 | 684 | 665 |
| Reactor Exit T gas (° C.) | 560 | 650 | 609 |
| Helium Feed sccm | 400 | 400 | 0 |
| HFC-236fa Feed sccm | 100 | 100 | 200 |
| Residence Time in Reaction | 2 | 2 | 5 |
| Zone (seconds) | | | |

Gas Chromatograph Results in Mole %

| | | | |
|---|---|---|---|
| CHCF₃ (HFC-23) | 0.0 | 0.0 | 0.1 |
| CHF═CF₂ (HFC-1123) | 0.0 | 0.0 | 0.1 |
| CF₃CH═CF₂ (HFC-1225zc) | 0.1 | 2.1 | 4.4 |
| CF₃CH₂CF₃ (HFC-236fa) | 99.7 | 97.6 | 95.3 |
| Other (<1%) | 0.2 | 0.2 | 0.3 |
| Conversion (%) | 0.3 | 2.4 | 4.7 |
| Yield (%) | 33 | 87.5 | 93.6 |

### Example 3

Reactor B (Nickel 200 reaction surface) is used. In this reactor the reactor temperature is the reactor center gas temperature (“Reactor Center Gas T” in Table 3). Runs A, B, and C are made at 800° C. with helium:reactant ratios of 0:1, 1:1, and 2:1, respectively. At these temperatures, higher than in Example 1, and at comparable reaction zone residence times, on the nickel surface, conversions are as high, and yields higher. In pyrolyses, higher temperatures generally lead to lower yields because of increased rates of undesirable side reactions giving unwanted byproducts. That this is not seen in Example 3 is testimony to the superiority of the nickel reaction surface to the nickel alloy reaction surface of Example 1. Further support for this conclusion is found in Run D, made at 850° C. with 4:1 helium dilution. Conversion is high at 76.9%, and the yield is 90.5%, the best of any of the Example 3 runs. Results are summarized in Table 3.

TABLE 3

| | | | |
|---|---|---|---|
| A | B | C | D |

Reactor Conditions, Feeds

| | | | | |
|---|---|---|---|---|
| Reactor Control T setting | 839 | 834 | 832 | 885 |
| (° C.) | | | | |
| Reactor Inlet T wall (° C.) | 812 | 806 | 804 | 853 |
| Reactor Middle T wall (° C.) | 831 | 826 | 824 | 877 |
| Reactor Exit T wall (° C.) | 808 | 805 | 804 | 855 |
| Preheat Gas T 1″ (° C.) | 658 | 666 | 669 | 707 |
| Reactor Inlet gas T 2″ (° C.) | 736 | 740 | 741 | 786 |
| Reactor Inlet gas T 3″ (° C.) | 779 | 780 | 781 | 829 |
| Reactor Center gas T 4″ | 800 | 800 | 800 | 850 |
| (° C.) | | | | |
| Reactor Exit gas T 5″ (° C.) | 800 | 800 | 799 | 851 |
| Reactor Exit gas T 6″ (° C.) | 776 | 777 | 777 | 829 |
| Exit gas T 7″ (° C.) | 738 | 741 | 740 | 791 |
| HFC-236fa Feed sccm | 200 | 200 | 200 | 200 |
| He Feed sccm | 0 | 200 | 400 | 800 |
| Residence Time in Reaction | 5 | 3 | 2 | 1 |
| Zone (seconds) | | | | |

GC Results in Mole %

| | | | | |
|---|---|---|---|---|
| CHCF₃ (HFC-23) | 4.1 | 2.5 | 2.0 | 2.5 |
| CHF═CF₂ (HFC-1123) | 0.7 | 1.0 | 1.0 | 1.4 |
| CF₃CH═CF₂ (HFC-1225zc) | 60.8 | 50.6 | 45.3 | 69.6 |
| CF₃CH₂CF₃ (HFC-236fa) | 28.2 | 37.7 | 50.2 | 23.1 |
| C₄H₂F₆ (HFC-1336) | 1.3 | 0.6 | 0.4 | 0.0 |
| Other (<1% produced) | 2.1 | 1.0 | 1.1 | 2.1 |
| Unknown | 2.8 | 6.6 | 0.0 | 1.2 |
| Conversion (%) | 71.8 | 62.3 | 49.8 | 76.9 |
| Yield (%) | 84.7 | 81.2 | 90.9 | 90.5 |

### Example 4

Reactor C (gold reaction surface). Like nickel, the gold surface gives high yields and therefore reduced side reactions producing unwanted byproducts. The inert gas diluent effect (reduction) on conversion is less on gold than on nickel or nickel alloy surfaces. At 800° C. (Runs A and B) conversions are lower than those of Runs B and C of Example 3 but the average yield is higher. Results are summarized in Table 4.

TABLE 4

| | | | | | |
|---|---|---|---|---|---|
| A | B | C | D | E | F |

Reactor Conditions, Feeds

| | | | | | | |
|---|---|---|---|---|---|---|
| Reactor Temp (° C.) | 800 | 800 | 700 | 700 | 600 | 600 |
| He Feed sccm | 15 | 20 | 15 | 20 | 15 | 20 |
| HFC-236fa Feed sccm | 10 | 5 | 10 | 5 | 10 | 5 |
| Residence Time in | 8 | 8 | 8 | 8 | 8 | 8 |
| Reaction Zone (seconds) | | | | | | |

GC Mole %

| | | | | | | |
|---|---|---|---|---|---|---|
| CHF₃ and CH₂F₂ | 1.9 | 1.9 | 0.1 | 0.1 | ND* | ND |
| CHF═CF₂ (HFC-1123) | 0.8 | 0.9 | ND | ND | ND | ND |
| CF₃CH₃ (HFC-143a) | 0.2 | 0.2 | ND | ND | ND | ND |
| CF₃CH═CF₂ (HFC- | 33.3 | 36.6 | 1.7 | 1.8 | 0.2 | 0.1 |
| 1225zc) | | | | | | |
| CF₃CHFCF₃ (HFC-227ea) | 0.2 | 0.2 | 0.1 | 0.1 | 0.1 | 0.1 |
| CF₃CH₂CF₃ (HFC-236fa) | 61.9 | 58.4 | 97.6 | 97.6 | 99.4 | 99.5 |
| Unknown | 0.6 | 0.5 | 0.3 | 0.1 | 0.2 | 0.1 |
| Conversion (%) | 38.1 | 41.6 | 2.4 | 2.4 | 0.6 | 0.5 |
| Yield (%) | 87.4 | 88.0 | 71 | 75 | 33 | 20 |

*ND = not detected

The Examples show the specificity of the pyrolysis according to this invention, which gives the product CF₃CH═CF₂ in good yield at good conversion with only small amounts of unwanted byproducts. Nickel is superior to nickel alloy as the reaction surface in giving higher yields of product. Gold is superior to nickel.

Conversions are low up to about 700° C., being good at 725° C. and above with no deterioration in performance even at 850° C.

What is claimed is:

1. Process comprising pyrolyzing CF₃CH₂CF₃ to CF₃CH═CF₂.

2. Process of claim 1 wherein said pyrolyzing is carried out to a single-pass conversion of said CF₃CH₂CF₃ of at least about 25%.

3. Process of claim 1 wherein said pyrolyzing is carried out at a temperature of at least about 700° C.

4. Process of claim 1 wherein said pyrolyzing is carried out for a reaction time of about 0.5 to 60 sec.

5. Process of claim 1 wherein said pyrolyzing is carried out to a single-pass yield of said CF₃CH═CF₂ of at least about 50%.

6. Process of claim 1 wherein said pyrolyzing is carried out in the presence of inert gas.

7. A process for the manufacture of 1,1,3,3,3-pentafluoropropene, comprising pyrolyzing 1,1,1,3,3,3-hexafluoropropane in the absence of dehydrofluorination catalyst in a reactor having a reaction zone.

8. The process of claim 7 wherein said reaction zone is substantially empty.

9. The process of claim 7 wherein said reaction zone is partially obstructed and has a free volume of at least about 80 percent.

10. The process of claim 1 wherein said pyrolyzing is carried out at a temperature of from about 700° C. to about 1000° C.

11. The process of claim 10 wherein said pyrolyzing is carried out at a temperature of from about 800° C. to about 900° C.

12. The process of claim 7 wherein the gas residence time in said reaction zone during said pyrolyzing is from about 0.5 to about 60 seconds.

13. The process of claim 7 wherein the gas residence time in said reaction zone during said pyrolyzing is from about 2 seconds to about 20 seconds, and wherein said pyrolyzing is carried out at a temperature of from about 800° C. to about 900° C. and a total pressure of about one atmosphere.

14. The process of claim 7 wherein the single-pass conversion of 1,1,1,3,3,3-hexafluoropropane during said pyrolyzing is at least about 25%.

15. The process of claim 7 wherein the single-pass yield of 1,1,3,3,3-pentafluoropropene during said pyrolyzing is at least about 50%.

16. The process of claim 7 wherein said pyrolyzing is carried out in the presence of unreactive diluent gas.

17. The process of claim 16 wherein said unreactive diluent gas is selected from the group consisting of nitrogen, argon, helium, trifluoromethane, and perfluorocarbons.

18. An azeotropic or azeotrope-like composition comprising hydrogen fluoride and 1,1,3,3,3-pentafluoropropene.

19. The composition of claim 18 containing about 60 mole percent of 1,1,3,3,3-pentafluoropropene.

20. A process for separating hydrogen fluoride from a first mixture comprising hydrogen fluoride and 1,1,3,3,3-pentafluoropropene wherein the amount of hydrogen fluoride in the first mixture is in excess of the amount of hydrogen fluoride in an azeotropic or azeotrope-like composition comprising hydrogen fluoride and 1,1,3,3,3-pentafluoropropene, comprising: distilling the first mixture to form a second mixture comprising an azeotropic or azeotrope-like composition comprising hydrogen fluoride and 1,1,3,3,3-pentafluoropropene; recovering the second mixture as a distillation column overhead stream, and; recovering hydrogen fluoride as a distillation column bottom stream.

21. A process for separating 1,1,3,3,3-pentafluoropropene from a first mixture comprising hydrogen fluoride and 1,1,3,3,3-pentafluoropropene wherein the amount of 1,1,3,3,3-pentafluoropropene in the first mixture is in excess of the amount of 1,1,3,3,3-pentafluoropropene in an azeotropic or azeotrope-like composition comprising hydrogen fluoride and 1,1,3,3,3-pentafluoropropene, comprising: distilling the first mixture to form a second mixture comprising an azeotropic or azeotrope-like composition comprising hydrogen fluoride and 1,1,3,3,3-pentafluoropropene; recovering the second mixture as a distillation column overhead stream, and; recovering 1,1,3,3,3-pentafluoropropene as a distillation column bottom stream.
